(19) United States
(12) Patent Application Publication
(10) Pub. No.: US 2006/0160198 A1
(43) Pub. Date: Jul. 20, 2006

(54) Method for the production of glycolic acid from ammonium glycolate by direct deammoniation

(76) Inventors: Xu Li, Newark, DE (US); George C. Fox, Wilmington, DE (US); Joseph J. Zaher, Newark, DE (US); Robert Dicosimo, Chadds Ford, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/315,709

(22) Filed: Dec. 21, 2005

Related U.S. Application Data
(60) Provisional application No. 60/638,148, filed on Dec. 22, 2004.

(51) Int. Cl. C12P 7/40

(52) U.S. Cl. 435/136; 562/577

(57) ABSTRACT

The present invention relates to a method for obtaining glycolic acid by direct deammoniation of ammonium glycolate. More specifically, water is removed from an aqueous solution of ammonium glycolate to produce the substantially anhydrous salt. Thermal salt cracking is then used to remove ammonia from the salt, producing a deammoniated product that is comprised of glycolic acid, oligomers of glycolic acid, ammonium salts of oligomers of glycolic acid, glycolamide, and unreacted ammonium glycolate. The deammoniated product can be further hydrolyzed to convert oligomers of glycolic acid to additional quantities of glycolic acid.

This application claims the benefit of U.S. Provisional Application No. 60/638,148, filed Dec. 22, 2004.

### FIELD OF THE INVENTION

This invention relates to a process for preparing glycolic acid from an aqueous solution of ammonium glycolate.

### BACKGROUND OF THE INVENTION

Glycolic acid (HOCH₂COOH; CAS Registry Number 79-14-1) is the simplest member of the α-hydroxy acid family of carboxylic acids. Its properties make it ideal for a broad spectrum of consumer and industrial applications, including use in water well rehabilitation, the leather industry, the oil and gas industry, the laundry and textile industry, and as a component in personal care products like skin creams. Glycolic acid also is a principal ingredient for cleaners in a variety of industries (dairy and food processing equipment cleaners, household and institutional cleaners, industrial cleaners [for transportation equipment, masonry, printed circuit boards, stainless steel boiler and process equipment, cooling tower/heat exchangers], and metals processing [for metal pickling, copper brightening, etching, electroplating, electropolishing]). New technology to commercially produce glycolic acid would be eagerly received by industry.

Various processes for preparing α-hydroxy acids are known. Enzymatic production of an α-hydroxy acid using the corresponding α-hydroxy nitrile as the starting material is known in the art. Examples of α-hydroxy acids produced via enzymatic hydrolysis of the corresponding nitriles include: glycolic acid, lactic acid, 2-hydroxyisobutyric acid, 2-hydroxy-2-phenyl propionic acid, mandelic acid, 2-hydroxy-3,3-dimethyl-4-butyrolactone, and 4-methylthiobutyric acid. These products are synthesized using microorganisms, such as those belonging to the genera Nocardia, Bacillus, Brevibacterium, Aureobacterium, Pseudomonas, Caseobacter, Alcaligenes, Acinetobacter, Enterobacter, Arthrobacter, Escherichia, Micrococcus, Streptomyces, Flavobacterium, Aeromonas, Mycoplana, Cellulomonas, Erwinia, Candida, Bacteridium, Aspergillus, Penicillium, Cochliobolus, Fusarium, Rhodopseudomonas, Rhodococcus, Corynebacterium, Microbacterium, Obesumbacterium and Gordona. (JP-A-4-99495, JP-A-4-99496 and JP-A-4-218385 corresponding to U.S. Pat. No. 5,223,416; JP-A-4-99497 corresponding to U.S. Pat. No. 5,234,826; JP-A-5-95795 corresponding to U.S. Pat. No. 5,296,373; JP-A-5-21987; JP-A-5-192189 corresponding to U.S. Pat. No. 5,326,702; JP-A-6-237789 corresponding to EP-A-0610048; JP-A-6-284899 corresponding to EP-A-0610049; JP-A-7-213296 corresponding to U.S. Pat. No. 5,508,181.)

Acidovorax facilis 72W (ATCC 55746) is characterized by aliphatic nitrilase (EC 3.5.5.7) activity, as well as a combination of nitrile hydratase (EC 4.2.1.84) and amidase (EC 3.5.1.4) activities. The gene encoding the A. facilis 72W (ATCC 55746) nitrilase has been cloned and recombinantly expressed (WO 01/75077 corresponding to U.S. Pat. No. 6,870,038; and Chauhan et al., Appl Microbiol Biotechnol, 61:118-122 (2003)). The A. facilis 72W nitrilase converts α-hydroxynitriles to the corresponding α-hydroxycarboxylic acids in high yield (U.S. Pat. No. 6,383,786), including glycolic acid (U.S. Pat. No. 6,416,980) (Equation 1).

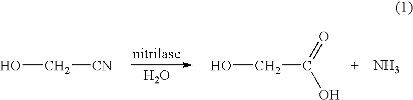

Enzymatic conversion of glycolonitrile to glycolic acid using an enzyme catalyst (nitrilase or a combination of a nitrile hydratase and an amidase) typically results in the production of an aqueous solution of ammonium glycolate. A variety of methods or processes can be used to obtain glycolic acid from the aqueous ammonium glycolate solution including, but not limited to concentration, crystallization, distillation, ion exchange (cationic and/or anionic), electrodialysis, reactive solvent extraction, polymerization, and alcoholysis. Many of these methods have limitations that are undesirable for industrial production such as 1) the use of low ammonium glycolate concentrations, 2) the generation of undesirable waste streams, and 3) the use of expensive and difficult to operate equipment (e.g., electrodialysis). Typically, methods such as cationic exchange and reactive solvent extraction require acidification of the carboxylic acid ammonium salt with a mineral acid to produce an aqueous solution of the carboxylic acid. Unfortunately, molar quantities of mineral salts are generated in the process, adding significant waste disposal costs. A method to reduce the amount of ammonium ion would reduce the amount of mineral salt generated.

One method that has been used to isolate carboxylic acids is reactive solvent extraction. This method has been reported to be useful for extracting lactic acid from aqueous solution (Wasewar et al., J. Biotechnol., 97:59-68 (2002)) and for extracting glycolic acid from water (Inci, I., Chem. Biochem. Eng. Q., 16:81-85 (2002)). Reactive extraction involves the use of a reactive organic solvent (i.e., an amine) to complex with the acid in the aqueous phase. The first step in the process typically involves acidification of the aqueous solution containing the salt of the desired acid. The acidified aqueous solution is then contacted with an organic solvent typically comprised of a reactive amine and one or more diluents. The reactive amine (typically a tertiary alkylamine such as Alamine® 336, Cognis Corp, Cincinnati, Ohio) reacts with the carboxylic acid forming an acid/amine complex that is preferentially soluble in the organic phase. Back extraction is then used to recover the acid from the organic phase. Unfortunately, molar quantities of mineral salts are generated in the process.

Another method to obtain glycolic acid from ammonium glycolate is thermal decomposition in the presence of an organic solvent. The solvent may act by protecting the glycolic acid from reactive ammonia (thereby decreasing the probability of amide formation) or may act as an organic reactive extraction solvent, thereby aiding in the separation of the acid (Meng et al., US 2004/0210087; herein incorporated by reference). Optionally, this method can also include an alcohol to act as an esterifying agent (which may be more soluble in the organic solvent). The organic solvent may be selected from the group consisting of tertiary alkylamines, alcohols, amides, ethers, ketones, phosphorus esters, phosphine oxides, phosphine sulfides, alkyl sulfides, and combinations thereof. Glycolic acid (or the corresponding ester) is then recovered from the organic solvent during a back extraction step. The recovered solvent can be recycled to the salt splitting reaction step. Unfortunately, solvent extraction/back extraction may be problematic as various immiscible fluids form complex physical mixtures that are difficult to separate.

Cockrem (U.S. Pat. No. 6,291,708) describes a process where an aqueous solution of an ammonium salt of an organic acid (i.e., ammonium lactate) is mixed with an alcohol and then heated rapidly under pressure to decompose the ammonium salt. Ammonia, water, and some of the alcohol (up to 90 wt %) are removed in the vapor stream producing free organic acid and/or the corresponding ester in the liquid product stream. The method is exemplified using ammonium lactate and requires the addition of an alcohol in the reaction mixture before thermal decomposition of the salt. Cockrem fails to address the separation of unreacted salts from the acid and ester.

WO 02/074403 A1 (corresponding to US 2003/0029711 A1 and US 2003/0029712 A1; herein incorporated by reference) describes a process for obtaining an organic acid from an organic acid ammonium salt, an organic acid amide, or an alkylamine organic acid complex. This method relies on the addition of an azeotroping agent to the aqueous feed stream prior to thermal decomposition. The vapor stream obtained by heating contains the azeotrope comprising the organic acid and the azeotroping agent. The components of the vapor stream can be separated using techniques known in the art, such as condensation, where the collected liquid stream can undergo additional distillation steps. This process is exemplified using a lactamide, ammonium lactate, or an alkylamine lactic acid complex and requires the addition of one or more chemicals (azeotroping agents) and a series of distillation steps. The presence of the alkylamine organic acid complex is the feed stream is indicative of a reactive solvent extraction step.

The problem to be solved, therefore, is the lack of a process for direct deammoniation of ammonium glycolate to form glycolic acid without the addition of additional chemicals.

### SUMMARY OF THE INVENTION

A process for obtaining glycolic acid is provided comprising:

(a) providing a feed stream comprising ammonium glycolate;

(b) removing free water from the feed stream to produce a substantially anhydrous salt of ammonium glycolate; and

(c) heating the product of step b) to a temperature of about 100° C. to about 140° C. under a vacuum sufficient to remove ammonia whereby a first product mixture comprising glycolic acid is produced.

In others aspects of this process, at least 90 wt %, 95 wt %, or 99 wt % of the free water in the feed stream (aqueous solution of ammonium glycolate) is removed. Methods to remove the free water in step (b) are selected from the group consisting of distillation, distillation at reduced pressure (vacuum distillation), evaporation, and lyophilization (freeze drying).

In another embodiment, the present process optionally includes the step of

(d) adding water to the first product mixture of step (c) to form a rehydrated first product mixture; said rehydrated first product mixture comprising glycolic acid, glycolamide, glycolic acid oligomers, glycolic acid oligomer ammonium salts, and unreacted ammonium glycolate; and

(e) heating the rehydrated first product mixture whereby a portion of the glycolic acid oligomers is hydrolyzed into free glycolic acid, wherein a second product mixture comprising glycolic acid is formed.

In one embodiment, glycolic acid can be isolated from the first or second product mixture using one or more techniques such as crystallization (e.g., US 2005/0020853 A1), ion exchange, electrodialysis, reactive solvent extraction, polymerization, distillation, and alcoholysis (U.S. 60/638,126). The remaining products (especially glycolamide) within either mixture can be subjected to further hydrolysis to produce glycolic acid.

A significant portion of the ammonium glycolate in the feed stream is converted into glycolic acid by the present process. As such, the amount of mineral salts produced during subsequent purification steps such as ion exchange is significantly reduced.

In another embodiment, the first or the second liquid product mixture requires no additional purification steps and may be used in a variety of industrial applications that do not require substantially purified glycolic acid.

### BRIEF DESCRIPTION OF THE FIGURES

The invention can be more fully understood from the Figures and the detailed description that together form this application.

FIG. 1 shows a general process of direct deammoniation (thermal salt cracking) to obtain glycolic acid from ammonium glycolate.

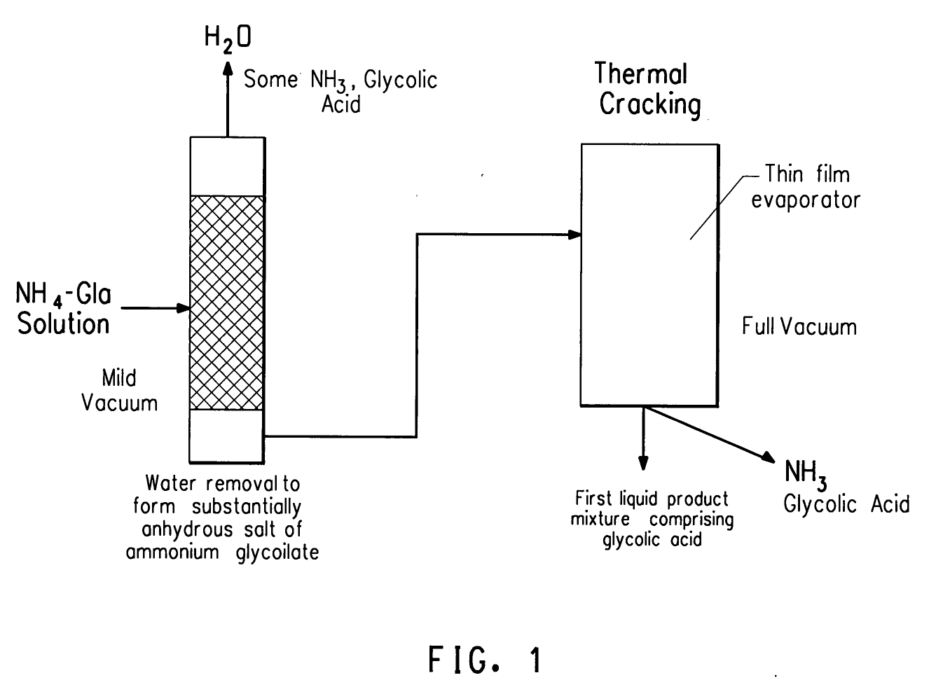

FIG. 2 shows the results of thermogravimetric analyzer—infrared spectrophotometer analysis (TGA-IR) for a substantially anhydrous ammonium glycolate salt. Thermal decomposition of the ammonium glycolate salt resulted in the generation of ammonia gas beginning around 96° C. Heating to temperatures above 140° C. generates additional water from condensation polymerization.

### DETAILED DESCRIPTION OF THE INVENTION

The stated problem has been solved by providing a direct deammoniation process for converting the ammonium glycolate into a first product mixture comprising glycolic acid. Specifically, free water is first removed from the aqueous ammonium glycolate feed solution to form the substantially anhydrous salt. Thermal decomposition of the ammonium salt (“thermal salt cracking”) is then used to remove the ammonia from the substantially anhydrous salt to form a product comprised of glycolic acid, oligomers of glycolic acid including glycolide, the ammonium salts of oligomers of glycolic acid, unreacted ammonium glycolate, and glycolamide. Removal of ammonium ion (via the generation of ammonia) produces a product mixture that can be subsequently processed to produce glycolic acid with significantly less waste generated. This is particularly true when the product mixture is acidified with a mineral acid to convert the majority of the remaining unreacted ammonium glycolate into glycolic acid. Additionally, the ammonia generated can be collected and recycled, helping to decrease to overall amount of waste generated.

In another aspect of the invention, the first product mixture is subsequently processed into a second product mixture, said process comprising the additional steps of 1) adding water to said first product mixture, thereby forming a rehydrated first product mixture; and 2) heating said rehydrated first product mixture whereby a portion of the glycolic acid oligomers in said rehydrated first product mixture is hydrolyzed into additional free glycolic acid, wherein a second product mixture is formed. In a further aspect, the glycolic acid in the first or second product mixture is isolated using one or more methods selected from the group consisting of reactive solvent extraction, crystallization, cationic ion exchange, anionic ion exchange, electrodialysis, alcoholysis, polymerization, and esterification.

In yet a further aspect, the first or second product mixture is used in a variety of applications that do not require a substantially purified form of glycolic acid.

In this disclosure, a number of terms and abbreviations are used. The following definitions apply unless specifically stated otherwise.

As used herein, the term “comprising” means the presence of the stated features, integers, steps, or components as referred to in the claims, but that it does not preclude the presence or addition of one or more other features, integers, steps, components or groups thereof.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably with 5% of the reported numerical value.

As used herein, “ATCC” refers to the American Type Culture Collection International Depository Authority located at ATCC, 10801 University Blvd., Manassas, Va. 20110-2209, USA. The “International Depository Designation” is the accession number to the culture on deposit with ATCC.

As used herein, “NRRL” refers to the Northern Regional Research Lab. The Northern Regional Research Lab has been renamed as the National Center for Agriculture, USDA Research (Peoria, Ill.).

As used herein, the term “glycolonitrile” is abbreviated as “GLN” and is synonymous with hydroxyacetonitrile, 2-hydroxyacetonitrile, hydroxymethylnitrile, and all other synonyms of CAS Registry Number 107-16-4.

As used herein, the term “glycolic acid” is abbreviated as “GLA” and is synonymous with hydroxyacetic acid, hydroxyethanoic acid, and all other synonyms of CAS Registry Number 79-14-1.

As used herein, the term “ammonium ion” refers to the cation having the formula NH₄⁺.

As used herein, the term “ammonium glycolate” is the ammonium salt of glycolic acid and is abbreviated as “NH₄GLA”.

The term “glycolamide” is the amide derived from the reaction of ammonia with glycolic acid and refers to all other synonyms of compounds having CAS Registry Number 598-42-5.

The term “glycolide” is the cyclic dimer of glycolic acid and is synonymous with 1,4-dioxane-2,5-dione and all other synonyms of CAS Registry Number 502-97-6. The term “oligomers of glycolic acid” include glycolide and linear oligomers of glycolic acid (dimers, trimers, etc.).

As used herein, the terms “direct deammoniation”, “thermal salt cracking”, “thermal salt decomposition”, “thermal decomposition”, and “salt cracking” refer to the process where a heat treatment is applied to the ammonium salt of the organic acid (i.e., ammonium glycolate) for a period of time to decompose the ammonium salt of the acid into the free organic acid and ammonia. In the present invention, thermal decomposition is used to produce primarily glycolic acid and ammonia. Various byproducts, such as glycolamide and oligomers of glycolic acid, may also be formed in the product mixture.

As used herein, the term “substantially anhydrous salt of ammonium glycolate” will refer to the resulting salt of ammonium glycolate (a liquid at room temperature) formed after removing at least about 90 wt %, preferably at least about 95 wt %, more preferably at least about 99%, and most preferably at least about 99.5 wt % of the free water in the feed stream (an aqueous solution comprising ammonium glycolate). As used herein, the term “free water” will refer to the water that is readily removed from the feed stream prior to thermal salt cracking, where some small but measurable amount of water may not be removed from the feed stream prior to thermal salt cracking (for example, water of hydration of the ammonium glycolate salt). As used herein, the term “molten salt of ammonium glycolate” will refer to the substantially anhydrous salt of ammonium glycolate that is thermally decomposed in the presence of a strong vacuum.

As used herein, the terms “aqueous solution of ammonium glycolate”, “aqueous solution comprising ammonium glycolate” and “feed stream” and will be used to describe the aqueous solution comprising ammonium glycolate. The feed stream is comprised of ammonium glycolate at a concentration of at least about 0.1 weight percent (wt %) to about 99.9 wt % ammonium glycolate. In another embodiment, the feed stream is comprised of at least about 10 wt % to about 75 wt % ammonium glycolate. In a further embodiment, the feed stream is comprised of at least about 20 wt % to about 50 wt % ammonium glycolate. The pH of the feed stream can be about 2 to about 12, preferably 5 to about 10, more preferably 6 to about 8. The pH can be adjusted as needed prior to use as a starting material in the present process.

As used herein, the term “partially deammoniated product” refers to the first product mixture or second product mixture produced by the present processes.

As used herein the term “first product mixture” or “first product mixture comprising glycolic acid” refers to the product obtained after thermally decomposing a substantially anhydrous salt of ammonia glycolate as described in the present processes. The first product mixture comprises glycolic acid, oligomers of glycolic acid, glycolamide, ammonium salts of oligomers of glycolic acid, and unreacted ammonium glycolate.

As used herein, the term “second product mixture” refers to the product obtained by the process of 1) adding water to said first product mixture to form a rehydrated first product mixture; and 2) heating said rehydrated first product mixture whereby a portion of the glycolic acid oligomers are hydrolyzed into free glycolic acid. Subsequent processing to obtain glycolic acid from the partially deammoniated products (i.e., first product mixture or second product mixture) produces significantly less waste in comparison to ammonium glycolate that was not thermally decomposed prior to processing.

As used herein, the term “rehydrated first product mixture” refers to the aqueous product obtained when water is added to the first liquid product mixture produced by the present processes.

As used herein, the terms “Acidovorax facilis” and “A. facilis” are used interchangeably and refer to Acidovorax facilis 72W (ATCC 55746). The term “E. coli SS1001” is used to described a recombinant E. coli host cell strain (deposited under ATCC PTA-1177) comprised of nitrilase activity derived from A. facilis 72W. The A. facilis 72W nitrilase is a particular robust catalyst that converts α-hydroxynitriles into the corresponding ammonium salt of the α-hydroxyacid using typical fermentation conditions (U.S. Pat. No. 6,416,980; hereby incorporated by reference in its entirety).

As used herein, the term “amidase” refers to an enzyme catalyst having the ability to hydrolyze an amide into the corresponding carboxylic acid and ammonia (E.C. 3.5.1.4). (Equation 2)

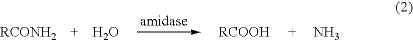

Genes encoding enzymes having amidase activity have also been cloned, sequenced, and expressed in recombinant organisms. For example, Azza et al., (FEMS Microbiol. Lett., 122:129 (1994)) disclose the cloning and over-expression in E. coli of an amidase gene from Brevibacterium sp. R312 under the control of the native promoter. Similarly, Kobayashi et al., (Eur. J. Biochem., 217:327 (1993)) teach the cloning of both a nitrile hydratase and amidase gene from R. rhodococcus J1 and their co-expression in E. coli. Wu et al. (DNA Cell Biol., 17:915-920 (1998); U.S. Pat. No. 6,251,650; herein incorporated by reference) report the cloning and overexpressing of a gene for amidase from Pseudomonas putida 5B (NRRL-18668) in E. coli. Payne et al. describe the isolation and recombinant expression of an amidase from Comamonas testosteroni 5-MGAM-4D (ATCC 55744) (US20050225116A1 and U.S. Ser. No. 10/997,893; each herein incorporated by reference).

Production of Aqueous Ammonium Glycolate “Feed Stream”

Enzymatic conversion of a nitrile (e.g., glycolonitrile) to a carboxylic acid typically results in the production of an aqueous solution of the ammonium salt of the carboxylic acid (e.g., ammonium glycolate) as the enzymatic reaction conditions are usually maintained at a pH where the predominant species is the ammonium salt of the desired carboxylic acid. In one embodiment, the aqueous solution of ammonium glycolate produced by an enzymatic process is used as the feed stream for the present process. In another embodiment, the feed stream is filtered to remove the biocatalyst (e.g., immobilized nitrilase catalyst) and other insoluble materials prior to being used as a feed stream in the present process.

Thermal Salt Cracking

The present process produces glycolic acid from the molten salt of ammonium glycolate without the addition of other chemicals (solvents, azeotroping agents, esterifying agents, tertiary amines, etc.).

The first step in the process is the removal of free water from a feed stream comprising an aqueous solution of ammonium glycolate, so that a substantially anhydrous ammonium glycolate salt is formed (the substantially anhydrous salt is a viscous liquid at room temperature (˜25° C.)). Methods of removing the free water from the aqueous reaction mixture are well-known in the art including, but not limited to distillation, vacuum distillation, lyophilization, and evaporation. In one embodiment, the free water is removed using vacuum distillation. Typically the aqueous solution of ammonium glycolate is heated to a temperature of about 40° C. to about 90° C., preferably about 40° C. to about 80° C. under a vacuum. The vacuum pressure may vary, but is typically about 0.5 mm Hg to about 700 mm Hg absolute pressure, preferably about 0.5 mm Hg to about 635 mm Hg absolute pressure, more preferably about 0.5 mm Hg to about 50 mm Hg absolute pressure. The length of time required to remove the free water may vary and can be determined by measuring the amount of free water removed. Typically, the amount of time required to form the substantially anhydrous salt is about 5 minutes to about 24 hours, preferably about 5 minutes to about 8 hours, more preferably about 1 hour to about 6 hours.

In one embodiment, the feed stream is heated to about 40° C. to about 80° C. using a vacuum of about 5 to about 25 mm Hg absolute pressure. In another embodiment, the vacuum applied is about 10 mm Hg absolute pressure and the temperature is about 40° C. to about 80° C. In yet another embodiment, the feed stream is heated to about 40° C. to about 70° C.; preferably about 40° C. to about 60° C., in vacuum of about 0.5 to 5 mm Hg absolute pressure; preferably at least about 1 mm Hg to about 5 mm Hg absolute pressure until a substantially anhydrous salt is formed. Optionally, a non-reactive gas (e.g., nitrogen) is used to aid in the removal of water when making the anhydrous ammonium glycolate salt. The amount of water removed can be measured using a variety of techniques well known in the art including, but not limited to weight loss (i.e., a 25 wt % ammonium glycolate solution should lose up to about 75% of its weight), changes in boiling temperature, and direct analysis of the of the vapor being removed. Some water may continue to evolve from the reaction mixture as side reactions (e.g., condensation polymerization) may generate some additional water.

The next step in the process involves heating the substantially anhydrous salt under a vacuum to a temperature sufficient to thermally decompose the ammonium salt into glycolic acid and ammonia as shown in Equation 3.
HOCH₂CO₂⁻NH₄⁺→NH₃+HOCH₂CO₂H (3)

The temperature used should be chosen so that thermal decomposition of the salt occurs while minimizing decomposition of the acid and/or minimizing unwanted side reactions that may generate undesirable byproducts such as glycolamide. Suitable vacuum pressures can be determined by one of skill in the art. An example of a typical vacuum range is about 0.5 to about 385 mm Hg absolute pressure. In one embodiment, the vacuum range is about 0.5 to about 80 mm Hg absolute pressure and the temperature is less than about 140° C. In another embodiment, the anhydrous salt is heated to a temperature of about 100° C. to about 140° C. under a vacuum of about 0.5 to about 1.5 mm Hg absolute pressure. In yet another embodiment, the substantially anhydrous salt is heated to a temperature of about 120° C. to about 140° C. under a vacuum of about 0.5 to about 5 mm Hg absolute pressure. In yet a further embodiment, the anhydrous salt is thermally decomposed at a temperature of about 120° C. to about 140° C. under a vacuum of about 0.5 to about 1.5 mm Hg absolute pressure. The absolute pressure during the thermal decomposition of the molten ammonium salt is in part dependent on the rate of generation of ammonia gas, and may be greater than the absolute pressure of the vacuum applied during the thermal decomposition. The thermal decomposition of the molten ammonium salt can use any evaporator design, however a wipe film evaporator is preferred.

The present process includes the step of heating the substantially anhydrous salt of ammonium glycolate. As used herein, the term “heating” or “heating period” refers to heat treatment step that includes both a time and temperature component. The heating period used to thermally decompose the molten ammonium glycolate salt into a product mixture (first liquid product mixture) comprising glycolic acid and may be adjusted depending upon the temperature and pressure used. The products of the reaction can be monitored (i.e. ammonia generated during thermal decomposition) to determine the amount of time necessary to obtain the desired product. In one embodiment, the amount of ammonia released is monitored to determine the length of time sufficient to produce the desired deammoniated product. In another embodiment, the heating period used to produce the deammoniated product is about 10 minutes to about 24 hours, preferably about 30 minutes to about 8 hours, more preferably about 1 hour to about 8 hours, and most preferably about 1 hour to about 6 hours.

Thermally decomposing the salt under the specified conditions converts a significant portion of the molten ammonium glycolate salt into glycolic acid and some additional byproducts such as glycolide (cyclic dimer of glycolic acid), linear polymeric forms of glycolic acid (typically dimers up to pentamers), the ammonium salts of linear polymeric forms of glycolic acid (typically dimers up to pentamers), and glycolamide. One of skill in the art can adjust the conditions used to thermally decompose the ammonium glycolate to optimize free glycolic acid production while minimizing undesirable side reactions, such as the production of glycolamide. The ammonia produced during thermal decomposition can be recovered and recycled.

Optionally, the aqueous ammonium glycolate solution is partially deammoniated to produce a deammoniated product that contains significantly less ammonium ion. This deammoniated product is particularly attractive for subsequent processing as less waste (mineral salts) is generated.

Side reactions, such as the reaction of ammonia with glycolic acid (Equation 4) or the polymerization of glycolic acid into oligomers release water.
HOCH₂CO₂H+NH₃→HOCH₂CONH₂+H₂O (4)

Use of steam stripping gas may be used to create a saturated headspace over the liquid anhydrous ammonium glycolate, creating conditions that thermodynamically suppress the undesirable side reactions. Optionally, a stripping gas (e.g., nitrogen, etc.) can be used to aid in the removal of ammonia from the anhydrous ammonium salt.

The first product mixture produced by thermal decomposition of the anhydrous salt of ammonium glycolate is generally comprised of glycolic acid, oligomers of glycolic acid (both linear and cyclic species such as glycolide), glycolamide, ammonium salts of oligomers of glycolic acid, and unreacted ammonium glycolate. In one aspect, the first product mixture may be further processed to chemically hydrolyze the oligomers of glycolic acid into free glycolic acid. This can be accomplished by first adding water to the first product mixture to produce a rehydrated product mixture. The rehydrated product mixture is subsequently heated for a period of time sufficient to hydrolyze at least a portion of the glycolic acid oligomers into free glycolic acid (monomeric) thereby forming a second product mixture. The amount of water added to the first product mixture may vary, but is typically about 5 wt % to about 95 wt %, preferably about 20 wt % to about 80 wt %, more preferably about 40 wt % to about 60 wt %, and more preferably about 50 wt % based on the total weight of the resulting rehydrated product mixture. As used herein, the term “heating the rehydrated product mixture” is used to describe a process wherein the rehydrated product mixture is heated to a temperature sufficient to hydrolyze at least a portion of the glycolic acid oligomers into free glycolic acid. In one embodiment, the heating (refluxing) conditions includes a temperature of about 90° C. to about 110° C., preferably about 100° C. to about 105° C. for a period of time from about 10 minutes to about 6 hours, preferably about 30 minutes to about 6 hours, more preferably about 1 hour to about 4 hours, and most preferably about 1.5 to about 3 hours.

Processes to Purify Glycolic Acid from the Deammoniated Liquid Product Mixtures

In one aspect, the glycolic acid produced by the present process can be isolated from the first or second product mixture using one or more techniques selected from the group consisting of, but not limited to crystallization, ion exchange, electrodialysis, reactive solvent extraction, polymerization, and alcoholysis (esterification followed hydrolysis of the carboxylic acid ester to release glycolic acid and the corresponding alcohol). After isolation of the glycolic acid from the reaction mixture, the remaining solution can be treated using a variety of methods or processes to convert the impurities (primarily the glycolamide and oligomers of glycolic acid) into glycolic acid. These methods can include, but are not limited to enzymatic conversion using an amidase, aqueous refluxing to hydrolyze the glycolamide into glycolic acid and ammonia, and combinations thereof. Optionally, the glycolic acid is not removed prior to converting the various byproducts into glycolic acid.

Subsequent purification steps may require the use of an aqueous solution, optionally acidified. Depending upon the subsequent processing step used, the deammoniated product (i.e. first or second liquid product mixture) may have water added and/or an acid added to the mixture prior to further processing. The resulting aqueous solution can be processed in a similar fashion as an aqueous ammonium glycolate solution that did not undergo deammoniation.

Various methods that may be used to purify glycolic acid from the partially deammoniated products (first or second product mixture) obtained by the present processes, are described below.

Ion Exchange (Cationic)

Ion exchange (cationic and/or anionic) typically requires acidification to favor formation of the acid over the corresponding ammonium salt. Cationic ion exchange uses a resin that traps the ammonium cation, allowing the glycolic acid, glycolamide, and glycolic acid oligomers to pass through.

Once the resin is saturated with ammonium ion, regeneration with an acid (e.g., sulfuric acid) will produce the byproduct ammonium sulfate salt. The cationic exchange can be performed in batches, using a simulated moving bed or carrousel. Selection of the resin may impact feed concentration, which may range from about 2 wt % salt to about 30 wt % ammonium glycolate. The regeneration acid used may range from about 0.5 wt % to about 20 wt %.

Ion Exchange (Anionic)

A similar process to cationic exchange except that a weak anionic resin is used. Passing an ammonium glycolate solution through the anionic resin will bind the glycolic acid while allowing the ammonium ion and ammonium glycolate to pass through. Once again, selection of the resin may impact feed concentration, which may range from about 2 wt % to about 30 wt % ammonium glycolate. Regeneration of the resin would typically use a weak acid.

Solvent Extraction (Reactive)

Reactive solvent extraction may also be used to obtain glycolic acid from an aqueous solution comprising ammonium glycolate (See Co-pending U.S. provisional patent application 60/638,128; herein incorporated by reference in its entirety). In the present invention, this may require the addition of water to the deammoniated product (first product mixture). In one approach to solvent extraction, an aqueous solution comprising ammonium glycolate is acidified (to a pH of about 3 or less, preferably a pH of about 2 or less) with a strong acid, such a sulfuric acid. The aqueous ammonium glycolate solution is typically comprised of about 5 wt % to about 70 wt % ammonium glycolate, preferably 5 wt % to about 40 wt % ammonium glycolate. An organic solvent is mixed with the acidified aqueous solution of glycolic acid, forming a 2-phase system. One or more components of the organic solvent reacts with the glycolic acid, forming a reaction complex that is preferentially soluble in the organic phase. A diluent may also be added to further enhance separation of the complex into the organic phase. The partition coefficient can be calculated by measuring the glycolic acid concentration in both the aqueous phase and the organic phase.

Organic bases and tertiary alkylamines have been shown to be useful for reactive extraction of carboxylic acids (Wasewar et al., J. Biotechnol. 97:59-68 (2002)). Tertiary alkyl amine extractants are particularly effective, with the equilibrium distribution coefficient (K_{D}) dependent upon the nature of the diluent used. One particularly useful tertiary amine is Alamine® 336 (a mixture of C8, C9, and C10 tertiary alkyl amines; Cognis Corp., Cincinnati, Ohio). Alaminee 336 in combination with methyl isobutyl ketone (MIBK) has been reported to be useful in extracting lactic acid from an aqueous solution prepared to resemble concentrations typically encountered during fermentative synthesis of lactic acid (Wasewar et al., supra).

A suitable organic extraction mixture for extracting glycolic acid is comprised of a mixture of Alamine® 336 with one or more diluents selected from the group consisting of methyl isobutyl ketone (MIBK), 1-octanol, 1-decanol, methylene chloride, 1-chlorobutane, chlorobenzene, chloroform, kerosene, toluene, mixed xylenes, and tributyl phosphate. In one embodiment, the organic phase extractant is comprised of Alamine® 336 in combination with one or more diluents selected from the group consisting of MIBK, 1-octanol, toluene, mixed xylenes, and kerosene. In another embodiment, the reactive organic solvent is comprised of about 50% (vol/vol) to about 95% (vol/vol) Alamine® 336, preferably about 65% (vol/vol) to about 95% (vol/vol) of the organic solvent mixture. The organic solvent is comprised of one or more diluents in a range of about 50% (vol/vol) to about 5% (vol/vol) diluent, preferably 35% (vol/vol) to about 5% (vol/vol) of the organic solvent mixture. In one embodiment, the mixed organic solvent is comprised of about 70% (vol/vol) Alaminee 336, about 10% (vol/vol) MIBK, and about 20% (vol/vol) kerosene. In another embodiment, the mixed organic solvent is comprised of about 90% (vol/vol) Alamine® 336 and about 10% (vol/vol) diluent selected from the group consisting of MIBK, 1-octanol, toluene, and mixed xylenes.

One of skill in the art can determine the optimal temperature of the organic phase extraction. In one embodiment, the extraction reaction is conducted at a temperature from about 5° C. to about 90° C., preferably 25° C. to about 75° C., and most preferably about 25° C. to about 50° C.

The amount of mixed organic solvent required to extract the glycolic acid from the acidified aqueous phase is dependent upon the degree of solvent loading. One of skill in the art can adjust the volume of the mixed organic solvent used to extract the glycolic acid depending upon the amount of glycolic acid present in the aqueous phase. The glycolic acid can be recovered from the organic phase by back extraction into water, forming an aqueous solution comprising glycolic acid.

Conversely, a reactive solvent extraction approach is also envisioned using a two-phase system where ammonia is complexed with reactive organic solvent. For example, a reactive organic solvent comprising at least one reactive aromatic carboxylic acid may be used to extract the ammonia from the aqueous phase. The ammonia will react with the aromatic carboxylic acid, forming a complex that is soluble in the organic phase while the small hydrophilic carboxylic acid (i.e., glycolic acid) remains in the aqueous phase. The glycolic acid could then be isolated from the aqueous phase by a well-known technique such as concentration or crystallization.

Another method to further purify glycolic acid from the partially deammoniated product produced by the present process is thermal decomposition in the presence of an esterifying agent. The solvent may act by protecting the glycolic acid from reactive ammonia (thereby preventing amide formation) or may act as an organic reactive extraction solvent, thereby aiding in the separation of the acid (Meng et al., US 2004/0210087; herein incorporated by reference in its entirety). Optionally, this method can also include an alcohol, thereby creating the ester (which may be more soluble in the organic solvent). The organic solvent may be selected from the group consisting of tertiary alkylamines, alcohols, amides, ethers, ketones, phosphorus esters, phosphine oxides, phosphine sulfides, alkyl sulfides, and combinations thereof. Glycolic acid (or the corresponding ester) is then recovered from the organic solvent (liquid phase) during a back extraction step with water.

Electrodialysis

Electrodialysis with bipolar membrane (EDBM) has been proposed for recovery of organic acids from their corresponding ammonium salt. For EDBM to work, the solutions must be conductive. For the ammonium salt of weak acids, the products of EDBM (organic acid and ammonium hydroxide) are very weak conductors resulting in high resistance of the solutions and low production rates. To offset this, a conductive salt (i.e., ammonium chloride) is added to the base loop (ammonium hydroxide stream). As the base concentration increases, ammonia can be stripped from the solution and the ammonium salt recycled to maintain conductivity.

The composition of the ammonium salt of the organic acid must be carefully monitored for multivalent cations such as calcium. These cations may precipitate by associating with hydroxyl groups and destroy the membranes. Concentrations of the multivalent cations are preferably below about 5 ppm, most preferably below about 1 ppm.

For example, a typical lab scale EDBM system can be set up with membranes suitable for ammonium salts. First, a recirculating base loop containing about 5 wt % ammonium chloride is established. An approximately 3 M ammonium glycolate recirculation loop is also established. A typical batch test run is conducted at constant current of about 0.5 to about 1.0 kA/m²; The circulation loops are maintained for about 1 hour to about 5 hours. As the EDBM proceeds, conductivity and pH in the ammonium glycolate loop decreases. Typically, an EDBM run under such conditions would be expected to convert at least about 80% of the ammonium glycolate into glycolic acid. The resulting glycolic acid/ammonium glycolate solution can be subsequently treated with a strong cationic ion exchange resin or other methods to complete the conversion.

Crystallization

Crystallization may be use to separate glycolic acid and/or glycolamide from the partially deammoniated product obtained by the present processes. Conditions such as temperature, pH, and solvent selection may be used to selectively crystallize the glycolic acid.

Alcoholysis (Esterification)

In order to overcome the problems associated with separating an ester from ammonium glycolate in a liquid matrix, alcoholysis using a heated alcohol vapor may be used (see co-pending U.S. provisional patent application 60/638,126; hereby incorporated by reference in its entirety). Ammonium glycolate will react with an alcohol vapor (e.g., heated methanol vapor) to form the corresponding ester while liberating ammonia with water as shown in Equation 5.
HOCH₂CO₂⁻NH₄⁺+R₂OH→HOCH₂CO₂R₂+NH₃+H₂O (5)

The heated alcohol vapor acts as both an esterifying agent and a stripping gas. The reaction produces a vapor product stream comprising glycolic acid ester vapor. The majority of unreacted ammonium glycolate is retained in the liquid phase.

The alcohol may include a C₁ to C₄ aliphatic alcohol or polyol, preferably a C₁ to C₄ alkyl alcohol esterifying agent, most preferably ethanol or methanol. The molar amount of alcohol stripping gas contacted with the ammonium glycolate solution may be from about 5 times to about 300 times the molar about of ammonium glycolate (molar ratio 5:1 to about 300:1), preferably about 5 to about 200 times the molar amount of ammonium glycolate (molar ratio of about 5:1 to about 200:1).

The alcohol vapor (e.g., methanol) temperature may vary, but is typically about 170° C. to about 300° C., preferably about 200° C. to about 260° C., and most preferably about 230° C. to about 260° C. The reactor pressure may vary but typically ranges from about 0 psig (0 kPa) to about 80 psig (˜552 kPa), preferably about 10 psig (˜69 kPa) to about 50 psig (˜552 kPa).

The glycolic acid ester (e.g., methyl glycolate) can be recovered from the vapor product stream using a rectifying distillation column or a partial condenser. In U.S. 60/638,126, a partial condenser (“hot condenser”) was used to recover methyl glycolate from the vapor product stream. In general, the partial condenser temperature if typically maintained at or below the boiling point of the glycolic acid ester but above the normal boiling point of the heated alcohol vapor. Typically, the partial condenser temperature is maintained at least 10° C. to about 100° C. below the normal boiling point of the respective glycolic acid ester. In one embodiment, the partial condenser temperatures should be about 50° C. to about 200° C., preferably about 50° C. to about 150° C. Careful control of the alcohol vapor temperature, the reactor pressure, and the partial condenser temperatures should selectively condense the glycolic acid ester (e.g., the methyl ester of glycolic acid) from the corresponding vapor product stream. The recovered glycolic acid ester can be hydrolyzed into glycolic acid and the corresponding alcohol. Techniques to hydrolyze esters to acids are known to those skilled in the art.

In one aspect, water may be added to the deammoniated product prior to alcoholysis. In another aspect, excess free water in the deammoniated product may be removed with mild temperatures and a mild vacuum prior to alcoholysis in order to concentrate the ammonium glycolate solution. In one embodiment, about 0.5 to about 5 moles of water per mole of ammonium glycolate remains, preferably about 1 to about 4 moles of water remains.

Alcoholysis may be conducted using an aqueous solution of ammonium glycolate having concentration of ammonium glycolate from about 5 wt % to about 99 wt %, preferably about 20 wt % to about 70 wt % ammonium glycolate.

Polymerization

The ammonium salt of a carboxylic acid comprising at least one hydroxyl group can undergo condensation polymerization to form dimers, oligomers, and polymers while liberating ammonia. The resulting polymers can be separated from the reaction mixture using any number of techniques known in the art. Once the polymers are separated from the reaction mixture, depolymerization can be used to obtain the free acid.

Esterification with Polyols

The ammonium salt of a carboxylic acid (ammonium glycolate) will react with polyols to form esters of the polyols and acid while liberating ammonia and water as shown in Equation 6.
2 HOCH₂CO₂⁻NH₄⁺+HOR₂OH→(HOCH₂CO₂)₂R₂+2NH₃+2H₂O (6)

A polyol is an organic molecule containing two or more hydroxyl groups including, but not limited to diols like ethylene glycol, propylene glycol, butanediol, hexanediol, triols like glycerol, and polyetherpolyols like polytetramethyl ether glycol, polypropylene oxide, and polyethylene oxide. Typically, this reaction is favored under conditions that rapidly remove ammonia and water. The free acid can then be obtained by hydrolysis of the esters.

Glycolamide Byproduct Deamidation

The formation of glycolamide is a byproduct that may be formed using the present processes. In the present processes, glycolamide is formed by the reaction of liberated ammonia with glycolic acid. However, glycolic acid can be produced from the glycolamide by reversing this reaction (chemical hydrolysis). The may be accomplished by hydrolyzing glycolamide with water. This process typically requires heating at elevated temperatures (Equation 7). Optionally, the hydrolysis reaction may also contain an acid catalyst.
HOCH₂CONH₂+H₂O→HOCH₂CO₂H+NH₃ (7)

Alternatively, glycolamide can also react with an alcohol or polyol to liberate the corresponding ester and ammonia as shown in Equation 8.
HOCH₂CONH₂+R₂OH→HOCH₂CO₂R₂+NH₃ (8)

The glycolic acid ester can be separated from the reaction mixture using a variety of techniques, such as distillation and/or solvent extraction. The isolated glycolic acid ester can be hydrolized into the corresponding acid and alcohol or polyol. Glycolic acid can be separated from the alcohol (or polyol) using a variety of techniques known in the art (i.e., distillation, crystallization, etc.)

Alternatively, any glycolamide produced can be treated with an enzyme having amidase activity to convert glycolamide to glycolic acid (Equation 2). In one aspect, water may be added to the deammoniated product (i.e., first liquid product mixture) prior to using an enzyme catalyst having amidase activity. Methods of converting amides to the corresponding acids using an amidase are known in the art (US 20040225116; US 20040023257; U.S. Pat. No. 6,617,139; U.S. Pat. No. 6,255,190; and U.S. Pat. No. 6,500,659; each herein incorporated by reference). Genes encoding enzymes having amidase activity have also been cloned, sequenced, and recombinantly expressed. For example, Azza et al., (FEMS Microbiol. Lett., 122:129 (1994)) disclose the cloning and over-expression in E. coli of an gene from Brevibacterium sp. R312 under the control of the native promoter. Similarly, Kobayashi et al., (Eur. J. Biochem., 217:327 (1993)) teach the cloning of both a nitrile hydratase and amidase gene from R. rhodococcus J1 and their co-expression in E. coli. Wu et al. (DNA Cell Biol., 17:915-920 (1998) and U.S. Pat. No. 6,251,650) report the cloning and overexpressing of a gene for amidase from Pseudomonas putida 5B in E. coli.

In one embodiment, the amidase activity (EC 3.5.1.4) of Comamonas testosteroni 5-MGAM-4D (ATCC 55744) or Pseudomonas putida 5B (NRRL-18668) is used to convert glycolamide to glycolic acid (U.S. Pat. No. 5,858,736; U.S. Pat. No. 5,922,589; and U.S. Ser. No. 10/977,893; all herein incorporated by reference in their entirety).

Use of the Product Mixtures Comprising Glycolic Acid

A described above, there are a variety of methods to isolate glycolic acid from the first or second product mixture produced by the present process. However, the product mixtures produced by the present processes may be used in a variety of industrial applications that do not require a more purified glycolic acid product. For example, it has been reported that compositions comprising one or more α-hydroxyacids, polymeric forms of α-hydroxyacids, poly-glycolic acid esters, glycolate ethers, ammonium and alkali metal salts of these hydroxyacids, and mixtures thereof are useful in removing metals and amine impurities from crude oil (US2004/0045875 A1; herein incorporated by reference).

Analysis of Glycolic Acid, Ammonium Glycolate, Glycolamide, Glycolide, and Linear Oligomers of Glycolic Acid

A variety of analytical methods can be employed to analyze the reactants and products produced using the present processes include high performance liquid chromatography (HPLC), gas chromatography (GC), and ion selective electrode (ISE) analysis. Glycolate, glycolamide, and glycolic dimer are analyzed by HPLC. Glycolide is analyzed by GC. Total ammonia is analyzed by ion electrode.

A. HPLC Conditions for Glycolic Acid Analysis (and Other Products):

Samples are diluted with water (as needed to be within HPLC detection range) and mixed 1:1 with 0.2 M n-propanol in water (HPLC external standard). HPLC Analytical Method: (BioRad HPX 87H ion exclusion column (BioRad, Hercules, Calif.), 300 mm×7.8 mm; 0.01 N H₂SO₄ mobile phase; 1.0 mL/min flow at 50° C.; 10 μL injection volume; Refractive Index (RI) detector and UV 210 nm, 20 min analysis time). HPLC Equipment: (Waters 2695 ‘Alliance’ Separations Module (Waters Corp, Milford, Mass.), 2410 Refractive Index Detector, 2487 Dual λ Absorbance Detector and Empower Pro Software). The retention times (RT) for various analytes are as follows:

| Analyte | RT (minutes) |
|---|---|
| Glycolic Acid Dimer | 6.74 |
| Glycolic Acid | 7.65 |
| Glycolamide | 11.00 |
| n-propanol | 16.26 (external standard) |

Oligomers of glycolic acid greater than dimer could not be analyzed by the present process, nor could the method distinguish between glycolic acid or glycolic acid dimer and their respective ammonium salts. Concentrations of carboxylic acids present were estimated from the difference in total concentration of carboxylic acids and their respective salts and the concentration of ammonium ion present in the sample.
B. Detection of Ammonia Using an Ion Selective Electrode:

Standards and samples are diluted with water (as needed to be within electrode detection range) and mixed (2% by volume) with ISA (Ion Strength Adjustor, Orion 951211 (Thermo Electron Corp., Waltham, Mass.)) to a pH above 11 (blue color indicator). Under constant stirring, immediate readings are taken with the ammonia electrode (Orion 9512). A standard curve is stored in the benchtop pH/ISE meter (Orion 720A+ (Thermo Electron Corp.) for direct concentration output in ppm NH₄⁺. Standards and the standards curve slope are checked approximately every 2 hours.

C. GC Analysis for Glycolide:

Raw samples were dissolved and diluted in acetone. Samples were subsequently analyzed by GC (Agilent Technologies 6890 System (Palo Alto, Calif.)) using the following column and conditions:

| Column: | J&W db-1701 capillary column | |
|---|---|---|
| | (Agilent Technologies) | |
| Nominal Length: | 30.0 | m |
| Nominal Diameter: | 530.00 | μm |
| Nominal Film Thickness: | 1.00 | μm |
| Carrier Gas: | Helium | |
| Total Flow: | 47.6 | mL/min |
| Injection Volume: | 0.5 | microliters |
| Inlet Split Ratio: | 6.788:1 | |
| Inlet Temperature: | 280° | C. |
| Oven Initial Temperature: | 50° | C. |
| Initial Time: | 1.00 | min |
| Ramp: | 10° | C./min |
| Final Temperature: | 220° | C. |
| Final Time: | 3.00 | min |

| | |
|---|---|
| Detector: | FID |

Ammonium glycolate solutions can be prepared by neutralizing glycolic acid with ammonium hydroxide.
D. Measurement of Products Released During Thermal Degradation of Aqueous Ammonium Glycolate Solutions

A thermogravimetric analyzer—infrared spectrophotometer (TGA-IR) was used to measure the gas products generated when an aqueous solution of ammonium glycolate (25 wt %) was heated over a temperature range of about 25° C. to about 500° C. at 5° C./min.

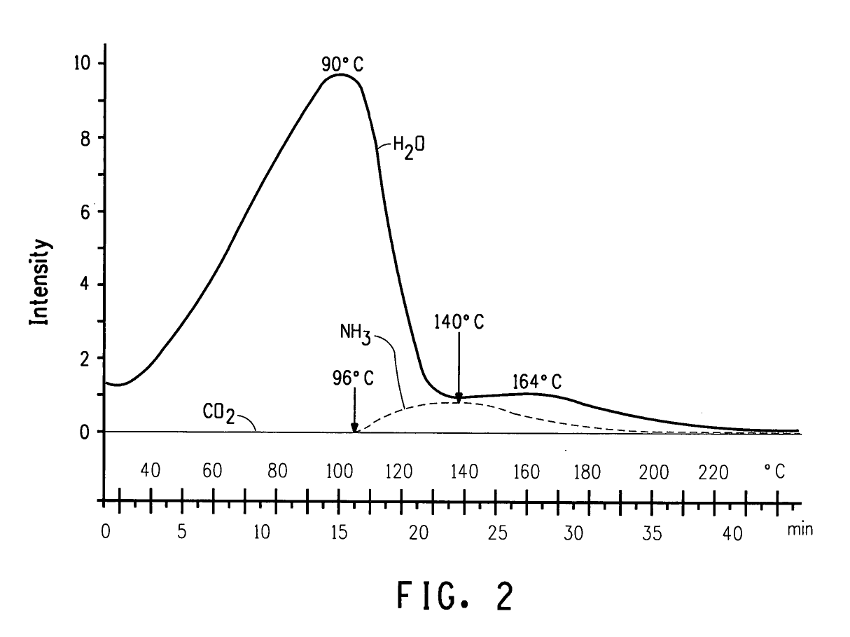

Water was evaporated from an aqueous ammonium glycolate solution (25 wt %). The resulting (substantially anhydrous) salt of ammonium glycolate was analyzed by TGA-IR. The peak in ammonia concentration in the gas phase was around 140° C., where some additional water was also generated (possibly from condensation polymerization) (FIG. 2). As such, it was determined that a maximum temperature of about 140° C. should be used to thermally decompose the ammonium glycolate salt while minimizing the degree of condensation polymerization.

Applicants specifically incorporate the entire contents of all cited references in this disclosure. Further, when an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### General Methods

The following examples are provided to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The abbreviations in the specification correspond to units of measure, techniques, properties, or compounds as follows: “sec” means second(s), “min” means minute(s), “h” means hour(s), “d” means day(s), “mL” means milliliters, “L” means liters, “mM” means millimolar, “M” means molar, “mmol” means millimole(s), “wt” means weight, “wt %” means weight percent, “g” means grams, “Hg” means mercury, “in Hg” means pressure as measured in inches of mercury, “mm Hg” means pressure in millimeters of mercury (absolute), “kPa” means kilopascals, “μg” means micrograms, “GC” means gas chromatography, and “HPLC” means high performance liquid chromatography.

### EXAMPLE 1

### Thermal Decomposition of Molten Ammonium Glycolate Salt Heated Up to ˜133° C. for 3.5 Hours

Approximately 54.65 g of 25 wt % ammonium glycolate solution was added to a 100-mL 3-neck flask, and water was removed by distillation. When the weight of liquid in the flask decreased to 13.63 g, the clear, syrup-like liquid was analyzed: 12% (mol/mol) of ammonium glycolate was converted to glycolamide, and 13% (mol/mol) of ammonium glycolate was converted to glycolic dimer (as combined glycolic acid dimer and ammonium glycolate dimer); glycolic acid (as combined glycolic acid and ammonium glycolate) and ammonium recovery were 70% (mol/mol) and 73% (mol/mol) respectively (yields calculated based on final molarity of products relative to final total molarity of ammonium glycolate and products). After a sample was removed for analysis by HPLC, Ion Electrode, GC, etc. (referred to as “sampling” from here on), 12.06 g of clear, viscous liquid remained in the flask. A vacuum of 254 mm Hg was imposed, and heating continued up to 133° C. over a period of 3.5 hours. Analysis of the resulting product showed 24% (mol/mol) conversion of starting ammonium glycolate to glycolamide, 32% (mol/mol) conversion of starting ammonium glycolate to glycolic dimer (as combined glycolic acid dimer and ammonium glycolate dimer), 20% (mol/mol) glycolic acid recovery (as, combined glycolic acid and ammonium glycolate) and 27% (mol/mol) of ammonium ion remaining. The combined yield of glycolic acid and glycolic acid dimer (separate from the ammonium glycolate and ammonium glycolate dimer present) was calculated to be at least approximately 9%.

### EXAMPLE 2

### Thermal Decomposition of Molten Ammonium Glycolate Salt Heated up to ˜140-150° C. for Approximately 6 Hours Followed by Hydrolysis of Glycolic Acid Oligomers

Approximately 54.82 g of 25 wt % ammonium glycolate solution was added to a 100-mL 3-neck flask, and water was removed by distillation. When the weight of liquid in the flask decreased 21.15 g, about 8 (mol/mol) % ammonia had been removed. After sampling, 19.91 g of clear, viscous liquid was left in the flask. A vacuum up to 74 mm Hg was imposed, and the temperature was raised to 140-150° C. within five hours and maintained for about an hour. The weight of residual liquid in the flask was 10.33 g. Analysis of this product indicated 24% (mol/mol) of ammonium glycolate was converted to glycolamide, 28% (mol/mol) of ammonium glycolate was converted to glycolic dimer (as combined glycolic acid dimer and ammonium glycolate dimer); glycolic acid (as combined glycolic acid and ammonium glycolate) and ammonium recovery were 20% and 11% (mol/mol), respectively. Approximately 7000 ppm of glycolide was also produced. After sampling, 9 g of the liquid was mixed with 9 g of water. The resulting solution was heated to about 105° C. and refluxed for two hours. No further reduction in ammonium ion was observed, and dimer and oligomer were converted to glycolic acid. Glycolamide concentration did not change significantly. Yield of glycolic acid dimer (as combined glycolic acid dimer and ammonium glycolate dimer) dropped from 24% (mol/mol) to 4% (mol/mol). Final glycolic acid recovery (as combined glycolic acid and ammonium glycolate) was 56% (mol/mol). Yield of glycolic acid was at least 45%.

### EXAMPLE 3

### Thermal Decomposition of Molten Ammonium Glycolate Salt Heated to ˜140-150° C. for Approximately 1 Hour Followed by Heating at 170° C.

Approximately 54.85 g of 25 wt % ammonium glycolate solution was added to a 100-mL 3-neck flask, and water was removed by distillation under vacuum (633 to 379 mm-Hg). When the weight of liquid in the flask decreased to 13.33 g, 10% (mol/mol) of ammonium glycolate was converted to glycolamide, and 13% (mol/mol) of ammonium glycolate was converted to glycolic dimer (as combined glycolic acid dimer and ammonium glycolate dimer); glycolic acid (as combined glycolic acid and ammonium glycolate) and ammonium recovery were 72% and 76% (mol/mol) respectively. After sampling, 11.98 g of a clear, viscous liquid remained in the flask. A vacuum of 127 mm-Hg was imposed, and temprature was maintained at 140-150° C. for an hour. Then the temperature was raised to 170° C. The liquid color turned brown within minutes. Analysis of this product indicated 29% (mol/mol) of ammonium glycolate was converted to glycolamide, 16% (mol/mol) of ammonium glycolate was converted to glycolic dimer (as combined glycolic acid dimer and ammonium glycolate dimer); glycolic acid (as combined glycolic acid and ammonium glycolate) and ammonium ion recovery were 30% and 16% (mol/mol), respectively. The combined yield of glycolic acid and glycolic acid dimer was at least approximately 22%.

### EXAMPLE 4

### Thermal Decomposition of Molten Ammonium Glycolate Salt Heated at 80° C. for Approximately 3 Hours Followed by Heating at 130° C. for 3 Hours

Approximately 29.1 g of 25% ammonium glycolate solution was produced from enzymatic hydrolysis of glycolonitrile using E. coli FM5/pNM18-H9 cells (See U.S. 60/638,176; hereby incorporated by reference in its entirety). The immobilized biocatalyst was decanted from the product solution. The solution was then added to a 100-mL 3-neck flask, and water was distilled off at 70-80° C. under vacuum (635-381 mm-Hg). When the weight of liquid in the flask decreased to 7.02 g, 3% (mol/mol) of ammonium glycolate was converted to glycolamide, and 12% (mol/mol) of ammonium glycolate was converted to glycolic dimer (as combined glycolic acid dimer and ammonium glycolate dimer); glycolic acid (as combined glycolic acid and ammonium glycolate) and ammonium recovery were 85% and 76% (mol/mol), respectively. After sampling, 4.2 g of a clear, viscous liquid remained in the flask. A vacuum of 127 mm-Hg was imposed and the temperature was maintained at 80° C. for three hours, then at 130° C. for three hours. Analysis of this product showed 26 (mol/mol) % conversion from glycolate to glycolamide, 28% (mol/mol) conversion from glycolate to glycolic dimer (as combined glycolic acid dimer and ammonium glycolate dimer), 44% (mol/mol) glycolic acid (as combined glycolic acid and ammonium glycolate) recovery, and 27% (mol/mol) of ammonium ion remained. Approximately 1.7 wt % of glycolide was also produced. The combined yield of glycolic acid and glycolic acid dimer was at least approximately 31

### EXAMPLE 5

### Thermal Decomposition of Molten Ammonium Glycolate Salt (Lyophilized) Heated to Approximately 80-90° C. for 6 Hours

Approximately 341.7 g of 40 wt % ammonium glycolate solution was frozen and lyophilized to remove water. Then 146.4 g of lyophilized ammonium glycolate was added to a flask, and heated to 80-90° C., then a vacuum of 50 mm-Hg was imposed. After six hours, no more ammonia was released. Final weight of the clear, viscous liquid was 104.4 g; 3% (mol/mol) of ammonium glycolate was converted to glycolamide, and 6% (mol/mol) of ammonium glycolate was converted to glycolic dimer (as combined glycolic acid dimer and ammonium glycolate dimer); glycolic acid (as combined glycolic acid and ammonium glycolate) and ammonium recovery were 66% (mol/mol) and 62% (mol/mol), respectively. Approximately 0.14 wt % of glycolide was also produced. The combined yield of glycolic acid and glycolic acid dimer was at least approximately 6%.

What is claimed is:

1. A process for obtaining glycolic acid comprising:
(a) providing a feed stream comprising ammonium glycolate;
(b) removing free water from the feed stream to produce a substantially anhydrous salt of ammonium glycolate; and
(c) heating the product of step b) to a temperature of about 100° C. to about 140° C. under a vacuum sufficient to remove ammonia whereby a first product mixture comprising glycolic acid is produced.

2. The process of claim 1 further comprising the steps of:
(d) adding water to the first product mixture of step (c) to form a rehydrated product mixture; said rehydrated product mixture comprising glycolic acid, glycolic acid oligomers, glycolamide, glycolic acid oligomer ammonium salts, and unreacted ammonium glycolate; and
(e) heating the rehydrated product mixture of step (d) under conditions whereby a portion of the glycolic acid oligomers are hydrolyzed into free glycolic acid, wherein a second product mixture comprising glycolic acid is formed.

3. The process of claim 1 or claim 2 additionally comprising the step of isolating the glycolic acid from the first product mixture of step (c) or the second product mixture of step (e).

4. The process of claim 3 wherein the glycolic acid is isolated using one or more processes selected from the group consisting of reactive solvent extraction, crystallization, cationic ion exchange, anionic ion exchange, electrodialysis, alcoholysis, polymerization, and esterification.

5. The process according to claim 1 wherein the water is removed in step (b) by vacuum distillation, evaporation, or lyophilization.

6. The process of claim 1 wherein the substantially anhydrous salt is heated to a temperature from about 100° C. to less than about 140° C. under a vacuum wherein the vacuum is about 0.1 mm Hg to about 350 mm Hg absolute pressure.

7. The process of claim 1 wherein at least 90% of the free water in the aqueous reaction solution is removed.

8. The process of claim 7 wherein at least 95% of the free water in the aqueous reaction solution is removed.

9. The process of claim 8 wherein at least 99% of the free water in the aqueous reaction solution is removed.

10. The process of claim 1 or claim 2 wherein the combined yield of glycolic and glycolic acid dimer is at least 6%.

11. The process of claim 1 wherein the feed stream of (a) is obtained by enzymatic hydrolysis of glycolonitrile.
